(19) United States
(12) Patent Application Publication
(10) Pub. No.: US 2021/0322661 A1
(43) Pub. Date: Oct. 21, 2021

(54) APHERESIS DEVICE CONTROLLER

(71) Applicant: Haemonetics Corporation, Boston, MA (US)

(72) Inventor: Melvin Tan, Edmonton (CA)

(21) Appl. No.: 17/271,448

(22) PCT Filed: Aug. 26, 2019
(86) PCT No.: PCT/US19/48123

§ 371 Date: Feb. 25, 2021

Related U.S. Application Data
(60) Provisional application No. 62/722,932, filed on Aug. 26, 2018.

(51) Int. Cl. A61M 1/34; A61M 1/36

(52) U.S. Cl. CPC A61M 1/3496; A61M 1/3693; A61M 2205/3561; A61M 2205/3576; A61M 2205/3553; A61M 2209/086; A61M 2205/70; A61M 2205/52; A61M 2205/502

(57) ABSTRACT

A controller for a blood processing device has a body that may dock and undock with a first blood processing device to connect the controller to the first blood processing device and disconnect the controller from the first blood processing device. A processor within the controller controls the first blood processing device when the controller is docked with the first blood processing device and remotely controls the first blood processing device when undocked. The controller also has a user interface that displays information regarding the first blood processing device and an ongoing apheresis procedure when the controller is docked to the first blood processing device and when the controller is undocked from the first blood processing device.

### PRIORITY

This PCT patent application claims priority from U.S. Provisional Application No. 62/722,932, filed Aug. 26, 2018, entitled “Apheresis Device Controller,” assigned attorney docket number 130670-09301 (formerly 1611/C93), and naming Melvin Tan as inventor, the disclosure of which is incorporated herein, in its entirety by reference.

### TECHNICAL FIELD

The present invention relates to operating an apheresis device, and more particularly to the removable components used to control and operate an apheresis device in a docked and undocked state and methods of the same.

### BACKGROUND ART

Apheresis is a procedure in which individual blood components can be separated and collected from whole blood withdrawn from a subject. An apheresis device is medical equipment designed and intended to perform an apheresis procedure. The apheresis device is a mechanical, electrical, and computerized device that is operated by a phlebotomist or qualified user by interacting with the input and output controllers built-into the device. The result of an operator input to the device is output as a visual display or audible feedback on the apheresis device controller.

As mentioned above, in some prior art systems, the apheresis device controller is built into the device. In such systems, the controller may be removable from the device only for servicing and replacement, but the controller may not operate the device when the controller is physically disconnected from the apheresis device itself. Because the apheresis device controller is built into the device and must always be physically connected to the device, the user must be within arm's reach of the apheresis device (e.g., when they are physically able to touch the device and the display) to control or operate the de [symbol] limits their mobility and ability to perform additional procedures from a distance.

Some prior art systems may use a separate portable computing device accessory to partially program the device and capture information output by the apheresis device. This portable computing device, however, does not control the device.

In cases where additional procedures need to be performed from a distance, the users will either delay recording information in the system, record information on paper to input the information into the system later, or employ a separate system that operates on a portable computing device for performing additional procedures. This requires additional equipment on the phlebotomy floor and potential connectivity between the apheresis system and the portable computing device system.

### SUMMARY OF THE EMBODIMENTS

In various embodiment of the invention, a portable computer such as a computer tablet is used as a controller to operate the apheresis device. The portable computer controller may operate the apheresis device while attached to the apheresis device, known as “docked,” or while unattached to the apheresis device, known as “undocked.”

The apheresis device can be wirelessly controlled from a remote location when the portable computer is undocked from the device. In this operating mode, the portable computer is operating as a wireless remote controller and the operator does not need to be within touching distance of the apheresis device to operate the device. Visual and audible feedback mechanisms continue to function in remote mode.

The portable computer can be re-docked to the apheresis device. In this mode, the controller functions as a normal attached apheresis device controller and retains the standard inputs, visual, and audible feedback mechanisms.

Docking and undocking the portable computer is a single step operation that attaches a connector on the apheresis device to a connector on the portable computer without requiring any additional connections, mounting, or manipulations of either the apheresis device or the portable computer. The act of attaching a connector on the apheresis device to a connector on the portable computer may be done either physically, electrically (e.g., via a USB port), and/or wirelessly (e.g., via a Bluetooth or other wireless technology [symbol] digital means.

In other embodiments of the invention, the portable computer used as a controller to operate the apheresis device may be used to control a plurality of devices. The operator can choose which device to operate in plurality mode and visual or audible feedback is given to the user for confirmation of the device that is currently being controlled by the portable computer.

When the portable computer is re-docked to the apheresis device, it begins to control the device to which it was docked. If the portable computer is docked to different apheresis device, the computer operates and controls the new device to which it is now docked and the previous device that the portable computer was controlling will no longer be controlled by the portable computer. When the user undocks the computer controller from the device it was recently docked, it continues to operate the recently docked apheresis device until such time the operator choses to control a different device.

In an additional embodiment of the invention, a new portable computer can be introduced into the system by docking the portable computer to an apheresis device. In such embodiments, the system may allow a new portable computer to be introduced into the system if the new portable computer is compatible with the system. Additionally or alternatively, the system also allows a portable computer/controller to be removed from the system so that it cannot operate any apheresis device in remote mode. The steps to add a new portable computer to the system must be followed to add the portable computer back into the system.

The portable computer may also be used to perform activities that are not related to the controlling of the apheresis device. For example, the system may service and record service information about the apheresis device or any other serviceable device. This activity can be performed while the portable computer is both docked and undocked from the apheresis device. Additionally or alternatively, the system may capture abnormal and exception events using the portable computer device. This activity can similarly be performed while the portable computer is both docked and undocked from the apheresis device.

In accordance with further embodiments, a controller for a blood [symbol] device includes a body, a processor and a user interface. The body may dock with a first blood processing device to connect the controller (e.g., a portable computer) to the first blood processing device, and may undock with the first blood processing device to disconnect the controller from the first blood processing device. The connection between the controller and the first blood processing device may be physical (e.g., the controller physically contacts the blood processing device), electrical (e.g., via a USB or similar electrical connection), and/or wirelessly (e.g., via Bluetooth or similar proximity/touch technologies). The processor may control the first blood processing device when docked with the first blood processing device and remotely control the first blood processing device when undocked. The user interface may display information regarding the first blood processing device and an ongoing apheresis procedure when the controller is docked to the first blood processing device and when the controller is undocked from the first blood processing device.

The controller and/or the processor may control at least a second blood processing device in addition to the first blood processing device. For example, the controller may control the second blood processing device when docked to the first blood processing device and/or when undocked from the first blood processing device. The controller may control the second blood processing device remotely. The user interface may allow the user to select which blood processing device to control. The body may dock with the second blood processing device to connect (e.g., physically, electrically or wirelessly) the controller to the second blood processing device. The controller may begin to automatically control the blood processing device to which it is docked.

The controller may also perform at least one additional function. For example, the controller may calibrate the blood processing device, calibrate additional components of a blood processing system, record procedure data, record and track procedure incidents, record donor activity, and record data related to sample collection. The controller may include a controller connector located on the body. The controller connector may connect with a device connector on the first blood processing device when the controller is docked with the first blood processing device. The body may fit within a docking portion of the first blood processing device such that the docking portion supports the body when the controller is docked to the first blood processing device. The docking portion may include [symbol] the body may fit within the recess when docked. In some embodiments, the controller may also have a data storage device that stores data relating to an apheresis procedure. The first blood processing device may be an apheresis device.

In accordance with further embodiments, a blood processing device includes a cabinet defining the structure of the blood processing device and housing one or more components of the blood processing device. The cabinet may also include a docking portion. The blood processing device may also have a blood component separation device for separating whole blood into one or more blood components, at least one pump configured to control a flow of whole blood and/or blood components through the blood processing device, and a controller. The controller may have a body configured to dock with the docking portion to connect the controller to the blood processing device. The connection between the controller and the first blood processing device may be physical (e.g., the controller physically contacts the blood processing device), electrical (e.g., via a USB or similar electrical connection), and/or wirelessly (e.g., via Bluetooth or similar proximity/touch technologies). The body may also undock with the blood processing device to disconnect the controller from the blood processing device. The controller may include a processor and a user interface. The processor may control the blood processing device when the controller is docked with the blood processing device and remotely control the blood processing device when the undocked. The user interface may display information regarding the blood processing device and an ongoing apheresis procedure when the controller is docked to the blood processing device and when the controller is undocked from the blood processing device

The controller may be a portable computer. The controller and/or the processor may control at least one additional blood processing device in addition to the blood processing device. The controller may control the additional blood processing device when docked to the blood processing device and/or when undocked from the blood processing device. The controller may control the additional blood processing device remotely. The user interface may allow the user to select which blood processing device to control. In some embodiments, the ability to control additional blood processing devices may be “locked out” (e.g., by an administrator) to prevent users from inadvertently controlling a [symbol] different/incorrect device.

In some embodiments, the body may dock with the additional blood processing device(s) to connect (e.g., physically, electrically, or wirelessly) the controller to the additional blood processing device(s). The controller may begin to automatically control the blood processing device to which it is docked. The controller may calibrate the blood processing device, calibrate additional components of a blood processing system, record procedure data, record and track procedure incidents, record donor activity, and record data related to sample collection.

In some embodiments, the blood processing device may include a blood processing device connector located on the cabinet and a controller connector located on the body of the controller. The controller connector may connect with the blood processing device connector when the controller is docked with the blood processing device. In embodiments that may constitute a physical or electrical connection with the controller, the blood processing device connector may be located within the docking portion. For example, the docking portion may include a recess and the body may fit within the recess when docked. In embodiments that make a wireless connection, the blood processing device connector may be within communication distance of the docking portion so that the controller may wirelessly connect. The controller may include a data storage device configured to store data relating to an apheresis procedure. The blood processing device may be an apheresis device.

In accordance with additional embodiments, a method for controlling an apheresis procedure on a blood processing device includes providing a blood processing device having a cabinet and a blood component separation device. The cabinet may define the structure of the blood processing device and may house one or more components of the blood processing device. The cabinet may also include a docking portion. The blood component separation device may separate whole blood into one or more blood components. The method may also include docking a controller with the blood processing device. The controller may have a (1) body configured to dock with the docking portion to connect the controller to the blood processing device, (2) a processor and (3) a user interface. The method may then control, using the controller and/or the processor, the blood p [symbol] device. Additionally or alternatively, the method may undock the controller from the blood processing device and remotely control the blood processing device using the undocked controller.

In other embodiments, the method may select, using the user interface, a second blood processing device, and remotely control the second blood processing device using the controller. Alternatively, the method may dock the controller with the second blood processing device and control the second blood processing device with the controller.

In further embodiments, the method may dock a second controller to the blood processing device and control the blood processing device with the second controller. Additionally, the method may select, using a second user interface on a second controller, the blood processing device. The method may then remotely control the blood processing device using the second controller.

### BRIEF DESCRIPTION OF THE DRAWINGS

The foregoing features of embodiments will be more readily understood by reference to the following detailed description, taken with reference to the accompanying drawings, in which:

FIG. 1 schematically shows a perspective view of a blood processing system in accordance with some embodiments of the present invention.

FIG. 2 schematically shows a top view of the blood processing system of FIG. 1, in accordance with some embodiments of the present invention.

FIG. 3 schematically shows a disposable set installed within the blood processing system of FIG. 1, in accordance with some embodiments of the present invention.

FIG. 4 schematically shows a portable controller docked with an apheresis device, in accordance with some embodiments of the present invention.

FIG. 5 shows the undocking ability of the apheresis device controller into a portable computer that can wirelessly control the apheresis device from a remote location, in accordance with some embodiments of the present invention.

FIG. 6 shows the apheresis device controller having ability to [symbol] apheresis device(s) when undocked, in accordance with additional embodiments of the present invention.

FIG. 7 shows the apheresis device controller being undocked and then being docked into a different apheresis device to control the new device, in accordance with various embodiments of the present invention.

FIG. 8 shows the apheresis device controller being able to perform non-apheresis procedure functions, in accordance with some embodiments of the present invention.

### DETAILED DESCRIPTION OF SPECIFIC EMBODIMENTS

In illustrative embodiments, controller for a blood processing device includes a body that may dock with a blood processing device to connect the controller to the blood processing device, and undock with the first blood processing device to disconnect the controller from the first blood processing device. The controller may have a processor that controls the blood processing device when docked or remotely when undocked. A user interface displays information regarding the first blood processing device and an ongoing apheresis procedure. The connections and disconnections between the controller and the blood processing device (e.g., the docking) may be physical, electrical and/or wireless.

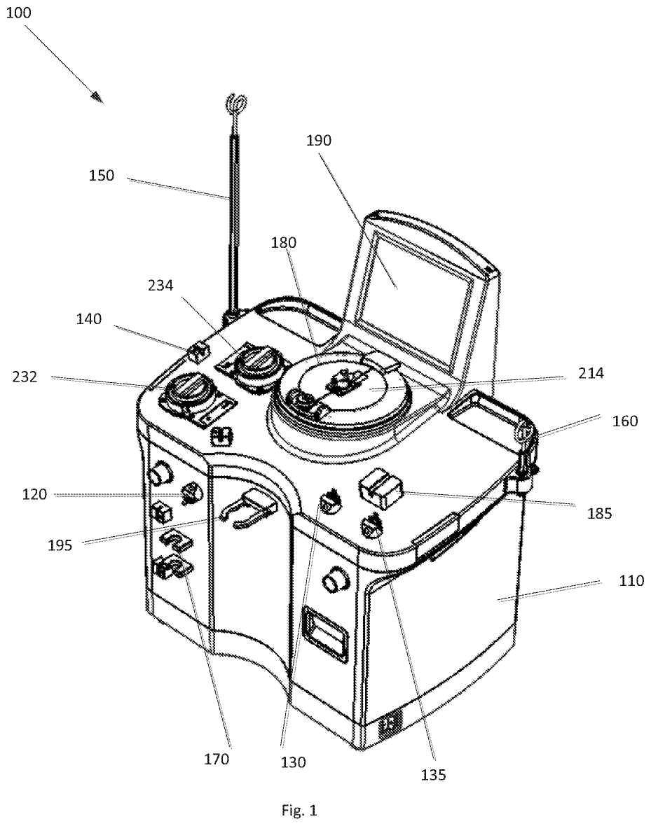

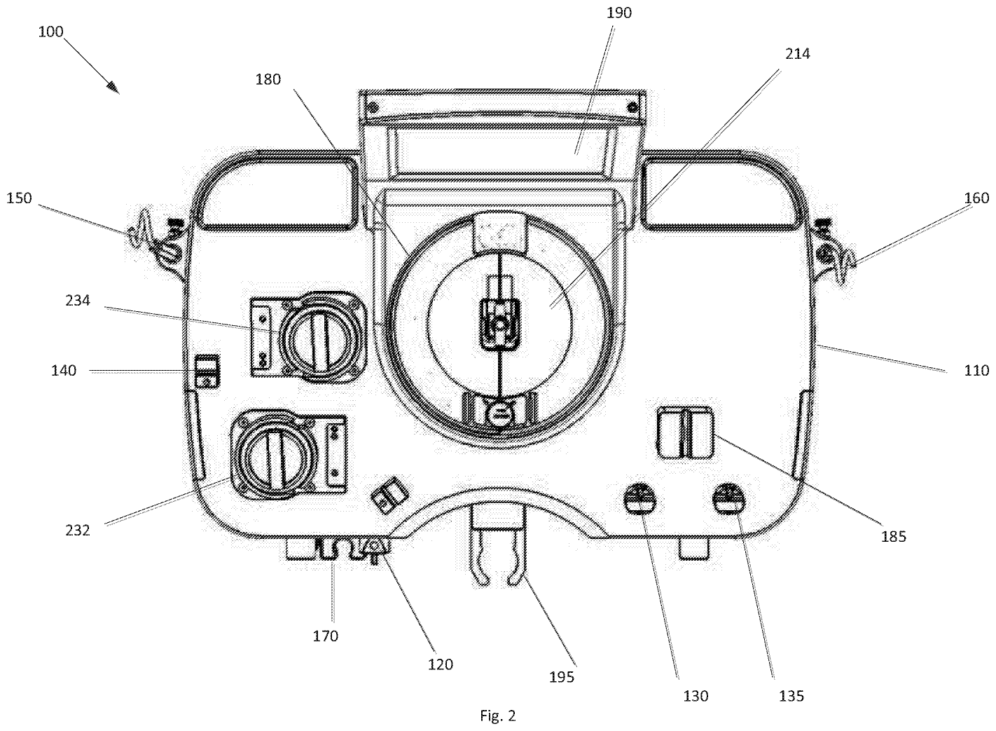

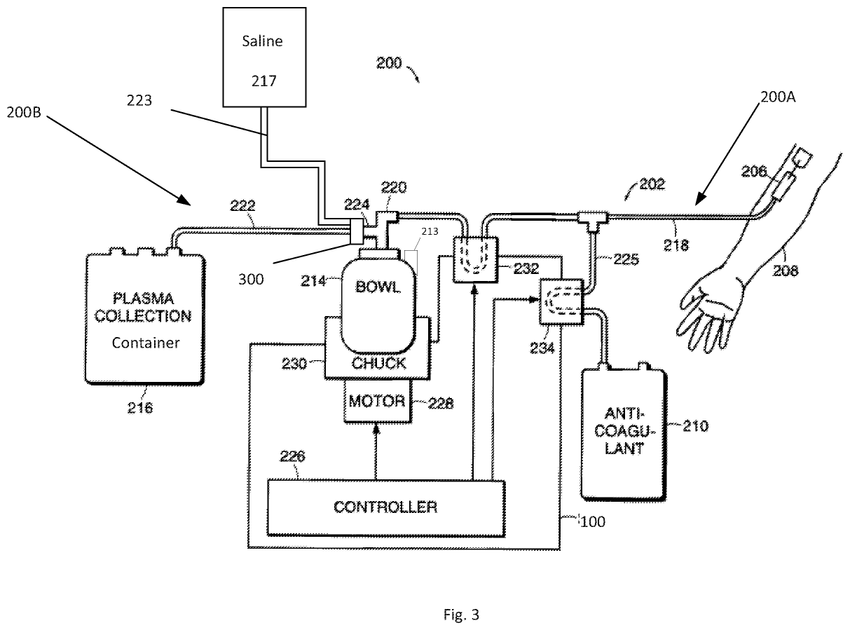

As shown in FIGS. 1 and 2, the blood processing system 100 includes a cabinet 110 that houses the main components of the system 100 (e.g., the non-disposable components). Within the cabinet 110, the system 100 may include a first/blood pump 232 that draws whole blood from a subject, and a second/anticoagulant pump 234 that pumps anticoagulant through the system 100 and into the drawn whole blood. Additionally, the system 100 may include a number of valves that may be opened and/or closed to control the fluid flow through the system 100. For example, the system 100 may include a donor valve 120 that may open and close to selectively prevent and allow fluid flow through a donor line 218 (e.g., an inlet line; FIG. 3), and a plasma valve 130 that selectively prevents and allows fluid flow through an outlet/plasma line 222 (FIG. 3). Some embodiments may also include a saline valve 135 that selectively prevents and allows saline to flow through a saline line 223.

To facilitate the connection and installation of a disposable set a [symbol] the corresponding fluid containers, the system 100 may include an anticoagulant pole 150 on which the anticoagulant solution container 210 (FIG. 3) may be hung, and a saline pole 160 on which a saline solution container 217 (FIG. 3) may be hung (e.g., if the procedure being performed requires the use of saline). Additionally, in some applications, it may be necessary and/or desirable to filter the whole blood drawn from the subject for processing. To that end, the system 100 may include blood filter holder 170 in which the blood filter (located on the disposable set) may be placed.

As discussed in greater detail below, apheresis systems 100 in accordance with embodiments of the present invention withdraw whole blood from a subject through a venous access device 206 (FIG. 3) using the blood pump 232. As the system 100 withdraws the whole blood from the subject, the whole blood enters a blood component separation device 214, such as a Latham type centrifuge (other type of separation chambers and devices may be used, such as, without limitation, an integral blow-molded centrifuge bowl, as described in U.S. Pat. Nos. 4,983,158 and 4,943,273, which are hereby incorporated by reference). The blood component separation device 214 separates the whole blood into its constituent components (e.g., red blood cells, white blood cell, plasma, and platelets). Accordingly, to facilitate operation of the separation device 214, the system 100 may also include a well 180 in which the separation device 214 may be placed and in which the separation device 214 rotates (e.g., to generate the centrifugal forces required to separate the whole blood).

To allow the user/technician to monitor the system operation and control/set the various parameters of the procedure, the system 100 may include a user interface 190 (e.g., a touch screen device) that displays the operation parameters, any alarm messages, and buttons which the user/technician may depress to control the various parameters. Additional components of the blood processing system 100 are discussed in greater detail below (e.g., in relation to the system operation).

FIG. 3 is a schematic block diagram of the blood processing system 100 and a disposable collection set 200 (with an inlet disposable set 200A and an outlet disposable set 200B) that may be loaded onto/into the blood processing system 100. The collection set 200 includes a venous access device 206 (e.g., a phlebotomy needle) for withdrawn [symbol] a donor's arm 208, a container of anti-coagulant 210, a centrifugation bowl 214 (e.g., a blood component separation device), a saline container 217, and a final plasma collection bag 216. The blood/inlet line 218 couples the venous access device 206 to an inlet port 220 of the bowl 214, the plasma/outlet line 222 couples an outlet port 224 of the bowl 214 to the plasma collection bag 216, and a saline line 223 connects the outlet port 224 of the bowl 214 to the saline container 217. An anticoagulant line 225 connects the anti-coagulant container 210 to the inlet line 218. In addition to the components mentioned above and as shown in FIG. 3, the blood processing system 100 includes a controller 226, a motor 228, and a centrifuge chuck 230. The controller 226 is operably coupled to the two pumps 232 and 234, and to the motor 228, which, in turn, drives the chuck 230. The controller 226 may be operably coupled to and in communication with the user interface 190. Alternatively, as discussed below, the interface 190 may be part of the controller 226.

In operation, the disposable collection set 200 (e.g., the inlet disposable set 200A and the outlet disposable set 200B) may be loaded onto/into the blood processing system 100 prior to blood processing. In particular, the blood/inlet line 218 is routed through the blood/first pump 232 and the anticoagulant line 225 from the anti-coagulant container 210 is routed through the anticoagulant/second pump 234. The centrifugation bowl 214 may then be securely loaded into the chuck 230. Once the bowl 214 is secured in place, the technician may install the outlet disposable set 200B. For example, the technician may connect a bowl connector 300 to the outlet 224 of the bowl 214, install the plasma container 216 into the weight senor 195, run the saline line 223 through valve 135, and run the plasma/outlet line 222 through valve 130 and the line sensor 185. Once the disposable set 200 is installed and the anticoagulant and saline containers 210/217 are connected, the system 100 is ready to begin blood processing.

As noted above, various embodiments of the apheresis device 100 discussed above have a controller 226. The controller 226 may control the operation of each of the components of the apheresis device 100 (e.g., the valves 122/130/135, pumps 232/234, separation device 214, motor 228, etc.) during processing and according to the program loaded onto the apheresis device 100. In some embodiments, the controller 226 may be located within the cabinet 110 of the system 100 and may not be removed during [symbol] (e.g., it is stationary except for during maintenance, repairs, etc.).

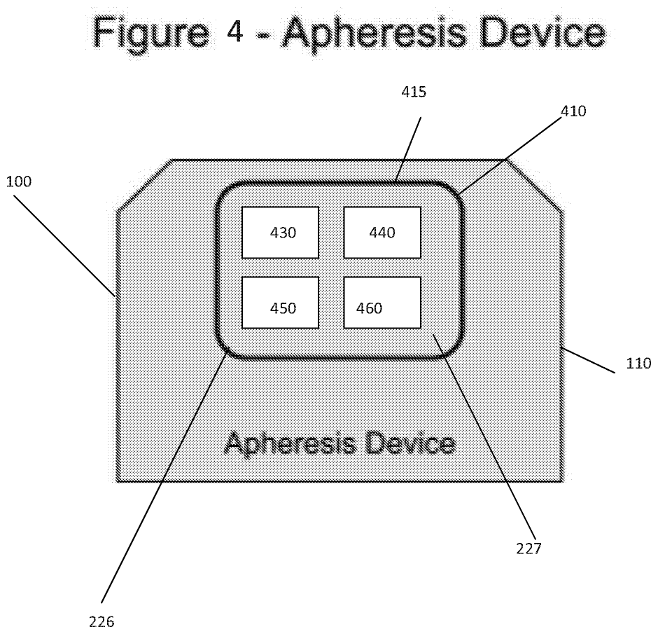

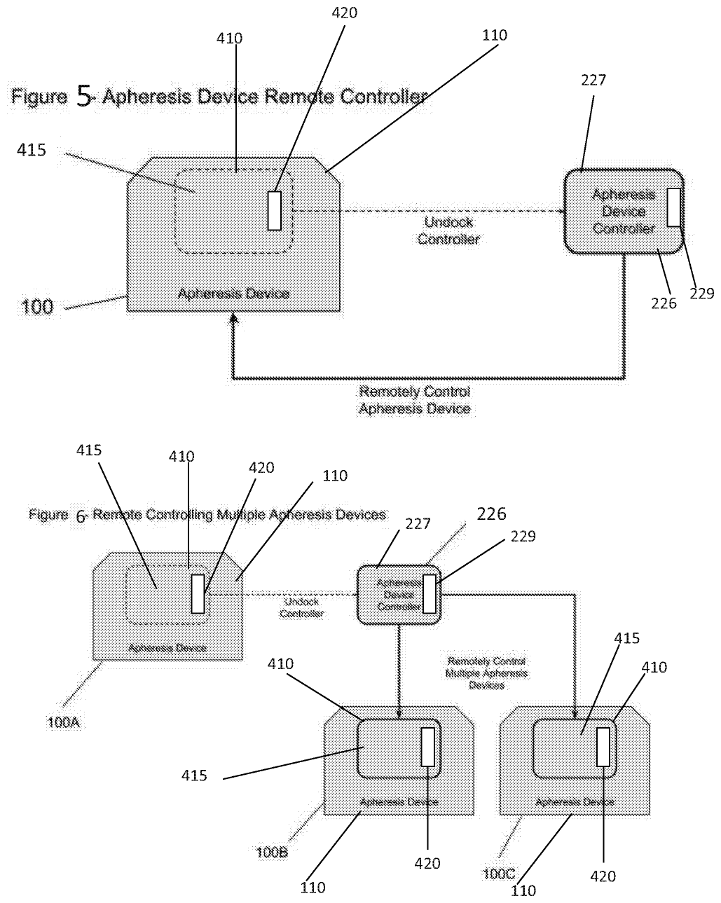

Alternatively, as shown in FIGS. 4 and 5, the controller 226 may be portable with respect to the apheresis device 100. For example, the controller 226 may be a portable computer that may be removed and taken away from the apheresis device 100. In such embodiments, the controller 226 may have a body 227 that docks with a docking portion 410 (e.g., a docking pod/station) within the apheresis device 100 to physically and, perhaps, electrically and/or wirelessly connect the controller 226 to the apheresis device 100. For example, the apheresis device may have a recessed portion 415 into which the controller 226 may be inserted to support the controller 226 within the apheresis device 100. Additionally, as shown in FIG. 5, the docking portion 410 may include an electrical connector 420 that connects with a corresponding connector 229 on the controller 226 to operatively connect the controller 226 and the apheresis device. In addition to providing a physical connection with the controller 226, the connectors 420/229 may also allow the apheresis device 100 to charge the controller 226 when the controller 226 is docked with the device 100.

It should be noted that, although the embodiment described above has a recessed portion 415 for the docking pod/station 410, other embodiments may not have such a recess and the controller 226 may simply be connected/docked to the apheresis device 100 by connecting the connector 420 on the device 100 with the connector 229 on the controller 226 (e.g., to electrically connect the controller 226 and the device 100). However, in either case, the controller 226 may connect to the apheresis device 100 in a single step. Additionally or alternatively, the controller 226 may wirelessly connect/dock to the blood processing device 100. In such embodiments, the controller 226 and blood processing device 100 may have Bluetooth modules (or similar wireless, proximity or touch technologies) located in or near the docking portion 410 and the controller 226 may wirelessly connect/dock to the blood processing device 100 once it is brought into close enough proximity to the docking portion 410 of the blood processing device 100.

The portable controller 226 may include a processor 430 that may control and monitor the apheresis device 100, and a memory 440 (e.g., a data storage device) for storing information relating the apheresis device(s) 100, the donor and/or any ongoing or past apheresis procedures performed by the apheresis device(s) 100. Additionally, t [symbol] 226 may include a communication port 450 and a controller interface/display 460 that provides information to the user/operator. In some embodiments, the display 460 may correspond to display 190 on the apheresis device (e.g., the interface 460 on the controller 226 acts as the interface 190 on the device 100).

As shown in FIG. 5, the controller 226 may also be “undocked” from the apheresis device 100 and may control the apheresis device 100 remotely. To that end, the user/operator may remove the controller 226 from the docking portion 410 on the apheresis device which, in turn, disconnects the controller connection 229 from the connector 420 on the apheresis device 100. For example, when there is a physical connection between the controller 226 and the apheresis device 100, the user may remove the controller from the recess 415 which may automatically disconnect the controller connection 229. Alternatively, if the connection is merely an electrical connection (e.g., via a USB port or similar wired connection), the user may manually disconnect the controller connection 229 to undock the controller 226. Lastly, if the connection is a wireless connection (e.g., via Bluetooth), the user may simply remove the controller 226 from the docking portion/area such that the wireless connections are no longer in close enough proximity

When the controller 226 is undocked from the apheresis device 100, the controller 226 operates as a remote controller and the operator does not need to be within touching distance of the apheresis device 100 (e.g., within touching distance of the interface 190 or any of the other components) to operate the apheresis device 100. When the controller 226 is docked (or re-docked) with the apheresis device 100, the controller 226 essentially functions as a normal attached device controller with the standard inputs and feedback mechanisms.

It is important to note that, even when undocked, the controller 226 may provide all the same functionality and control as when in it is docked with the apheresis device 100 (e.g., it may provide the same control and information as a controller that is integral with the apheresis device 100). For example, the controller 106 may show, via the display 460 on the controller 226, the progress and status of the apheresis device 100 and procedure. Additionally, the controller 226 may be configured with an audible and/or tactile alarm corresponding to an error message from apheresis device 100. By provid [symbol] control of the apheresis device 100, the controller 226 may replace, augment, or reproduce a controller that is integral with the apheresis device 100 and/or the built-in display 190 on the apheresis device.

In addition to controlling the device 100 to which the controller 226 is docked (or was previously/originally docked with), as shown in FIG. 6, the controller 226 may monitor and/or control a number of other apheresis devices 100, for example, within a donation center. To that end, the controller 226 may include a plurality mode that displays the various apheresis devices 100 (devices 100A/B/C) that it is in communication with and may control. The user may then select the desired apheresis device 100 to allow the user to control and/or monitor the selected apheresis devices 100A/B/C. The controller 226 may display the possible apheresis devices 100A/B/C to control in a number of different ways. For example, the controller 226 may simply display a list of possible devices 100A/B/C to control. In some cases, the list of devices 100A/B/C may be supplemented with additional information such as donor photo, device ID, or the likes for helpful and easy identification of which device to take control. Alternatively, the controller 226 may display an image of the floor plan of the donation center and the locations of each of the apheresis devices 100A/B/C, and the user may simply touch the apheresis device that they wish to control. It should be noted that, in some embodiments, the ability to control additional blood processing devices may be “locked out” (e.g., by an administrator) to prevent users from inadvertently controlling a different/incorrect device.

It should be noted that, when the controller 226 is re-docked with the apheresis device 100 (or docked with a new apheresis device), the controller 226 may begin to automatically control the device 100 to which it was docked (e.g., it does not require the user to select the particular device). For example, if the controller 226 is currently connected to and/or remotely controlling device 100B and is then docked with device 100A, the controller 226 may automatically switch to controlling device 100A (e.g., the device to which it is now docked). Similarly, if the controller 226 is docked to a different apheresis device (e.g., it is subsequently docked with device 100C), the controller 226 is able to control the new device (e.g., device 100C) to which it is now docked. Additionally or alternatively, when the user undocks the controller 226 from the device it was recently docke [symbol] controller 226 may continue to operate the recently docked apheresis device 100 until such time as the operator choses to control a different device (e.g., if the device is undocked from device 100A, it will continue controlling device 100A until the user selects a new device to control).

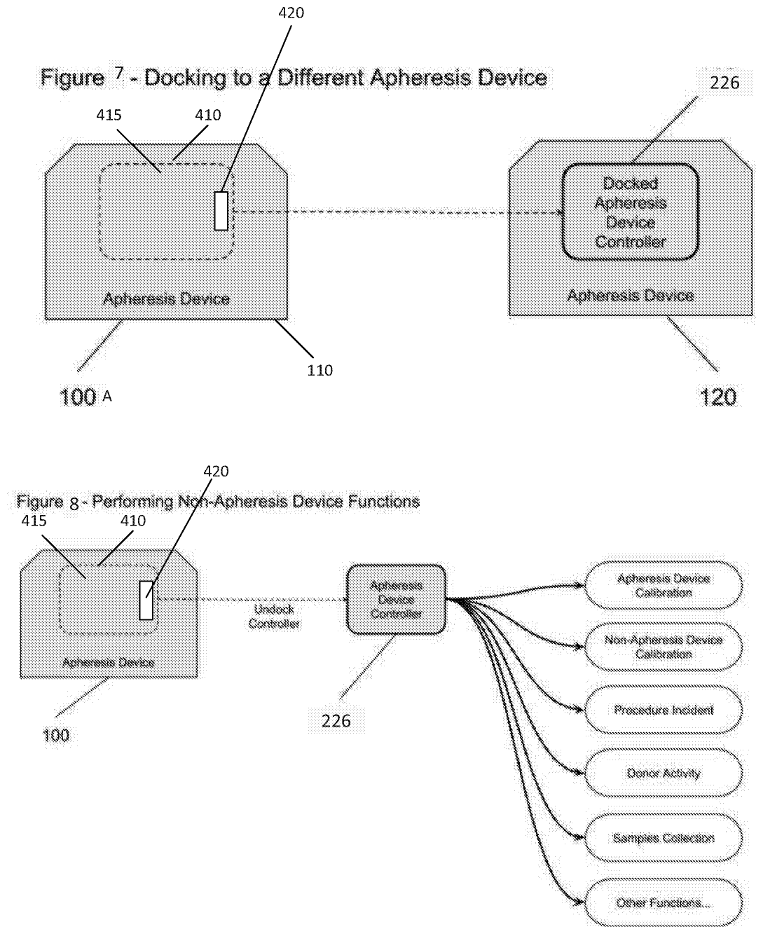

Eallch of the apheresis devices 100A/B/C may be compatible with a number of different controllers 226. Therefore, a new controller 226 may be introduced into the system simply by docking the new controller 226 to one of the devices 100A/B/C and/or selecting the device 100A/B/C on the new controller 226. Therefore, if a new user enters the donation center or the first controller 226 begins to malfunction, a new controller 226 may be easily introduced to ensure that the blood processing procedures can continue as planned. Similarly, as shown in FIG. 7, because the controllers 226 are compatible with a number of apheresis devices, each of the controllers 226 may be connected to/docked with different apheresis devices (e.g., a second apheresis device 120) such that the various controllers 226 may be interchangeably used to control some or all of the apheresis devices within the donation center.

In some embodiments, there may be security measures in place to prevent one user/controller 226 from taking control over an apheresis device 100 that is currently under control of another user/controller 226. For example, if an apheresis device 100 is currently being controlled by another controller 226 and a new controller 226 tries to connect to the device 100 to take control, the device 100 and/or the new controller 226 may send a message to or otherwise alert the original controller 226 of the request of the new controller 226 to connect to/control the device 100. The user of the currently connected controller 226 may then allow and/or not allow the new controller 226 to take control (e.g., by pressing an “allow” or “not allow” button displayed on the controller 226). Additionally or alternatively, the new control may provide an elevated privileged override capability to connect to/control the device 100 from the original controller 226 if necessary.

In addition to controlling any number of apheresis devices 100A/B/C, as shown in FIG. 8, the controller(s) 226 may perform additional activities that are not necessarily related to controlling the apheresis device(s) 100. For example, the controller(s) 226 may service and record service information about the apheresis devices 10 [symbol] other serviceable device related to the blood processing procedure. This activity can be performed while the controller 226 is both docked and undocked from the apheresis device 226. Additionally or alternatively, the controller 226 may capture abnormal and exception events (e.g., procedure events) during the apheresis procedure. Also when not performing an apheresis procedure, the controller 226 may be used to help calibrate various devices used in connection with the blood processing procedure (including the apheresis device 100 and other non-apheresis devices), and monitor and record donor activity and sample collection. These activities can be performed while the controller 226 is both docked and undocked from the apheresis device(s) 100. It should be noted that the ability to perform these additional functions may be user configurable (e.g., the user may elect to allow the controller 226 to have this functionality or not have this functionality).

Furthermore, the controller(s) 226 may continue to perform additional activities that are not necessarily related to controlling the apheresis device even if the apheresis device 100 is powered off. This allows the apheresis device 100 to be serviced while powered off for safety reasons, but the controller(s) 226 will still provide the necessary functions to service the device 100. The work done in the controller(s) 226 while the apheresis device 100 is powered off is maintained locally in the controller(s) 100 and optionally transmitted wirelessly to a remote computer system (e.g., using the communication port 450), wirelessly to another powered apheresis device 100, or transmitted later to the powered off apheresis device 100 when it is powered on again.

In addition to the individual apheresis devices 100, the controller(s) 226 may operatively connect with a centralized data system via a wireless communication system and/or using the communications port 450. The centralized data system may be a part of the donation center network, a local-area network, an internet/cloud-based network, or a network connecting multiple donation centers. The centralized data system may include a database and may provide information relating to the donor records and the apheresis procedure, for example, to the controller 226. During an apheresis procedure, the controller 226 may show on the display 460, for example, the donor information and device information/identification. Additionally, the controller 226 may upload information regarding the monitor [symbol] apheresis procedures to the centralized data system.

It should be noted that terms such as “controller,” “processor” and “server” may be used herein to describe devices that may be used in certain embodiments of the present invention and should not be construed to limit the present invention to any particular device type or system unless the context otherwise requires. Thus, a system may include, without limitation, a client, server, computer, appliance, or other type of device. Such devices typically include one or more network interfaces for communicating over a communication network and a processor (e.g., a microprocessor with memory and other peripherals and/or application-specific hardware) configured accordingly to perform device and/or system functions. Communication networks generally may include public and/or private networks; may include local-area, wide-area, metropolitan-area, storage, and/or other types of networks; and may employ communication technologies including, but in no way limited to, analog technologies, digital technologies, optical technologies, wireless technologies, networking technologies, and internetworking technologies

The various components of the control program may be implemented individually or in combination. For example, each component may be implemented or a dedicated server or a set of servers configured in a distributed manner

It should also be noted that devices may use communication protocols and messages (e.g., messages created, transmitted, received, stored, and/or processed by the system), and such messages may be conveyed by a communication network or medium. Unless the context otherwise requires, the present invention should not be construed as being limited to any particular communication message type, communication message format, or communication protocol. Thus, a communication message generally may include, without limitation, a frame, packet, datagram, user datagram, cell, or other type of communication message. Unless the context requires otherwise, references to specific communication protocols are exemplary, and it should be understood that alternative embodiments may, as appropriate, employ variations of such communication protocols (e.g., modifications or extensions of the protocol that may be made from time-to-time) or other protocols either known or developed in the future.

It should also be noted that logic flows may be described hereir [symbol] demonstrate various aspects of the invention, and should not be construed to limit the present invention to any particular logic flow or logic implementation. The described logic may be partitioned into different logic blocks (e.g., programs, modules, interfaces, functions, or subroutines) without changing the overall results or otherwise departing from the true scope of the invention. Often times, logic elements may be added, modified, omitted, performed in a different order, or implemented using different logic constructs (e.g., logic gates, looping primitives, conditional logic, and other logic constructs) without changing the overall results or otherwise departing from the true scope of the invention.

The present invention may be embodied in many different forms, including, but in no way limited to, computer program logic for use with a processor (e.g., a microprocessor, microcontroller, digital signal processor, or general purpose computer), programmable logic for use with a programmable logic device (e.g., a Field Programmable Gate Array (FPGA) or other programmable logic device (PLD)), discrete components, integrated circuitry (e.g., an Application Specific Integrated Circuit (ASIC)), or any other means including any combination thereof. In some embodiments of the present invention, predominantly all of the described logic is implemented as a set of computer program instructions that is converted into a computer executable form, stored as such in a computer readable medium, and executed by a microprocessor under the control of an operating system.

Computer program logic implementing all or part of the functionality previously described herein may be embodied in various forms, including, but in no way limited to, a source code form, a computer executable form, and various intermediate forms (e.g., forms generated by an assembler, compiler, linker, or locator). Source code may include a series of computer program instructions implemented in any of various programming languages (e.g., an object code, an assembly language, or a high-level language such as FORTRAN, C, C++, JAVA, or HTML) for use with various operating systems or operating environments. The source code may define and use various data structures and communication messages. The source code may be in a computer executable form (e.g., via an interpreter), or the source code may be converted (e.g., via a [symbol] assembler, or compiler) into a computer executable form.

The computer program may be fixed in any form (e.g., source code form, computer executable form, or an intermediate form) either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), a PC card (e.g., PCMCIA card), or other memory device. The computer program may be fixed in any form in a signal that is transmittable to a computer using any of various communication technologies, including, but in no way limited to, analog technologies, digital technologies, optical technologies, wireless technologies, networking technologies, and internetworking technologies. The computer program may be distributed in any form as a removable storage medium with accompanying printed or electronic documentation (e.g., shrink wrapped software), preloaded with a computer system (e.g., on system ROM or fixed disk), or distributed from a server or electronic bulletin board over the communication system (e.g., the Internet or World Wide Web).

Hardware logic (including programmable logic for use with a programmable logic device) implementing all or part of the functionality previously described herein may be designed using traditional manual methods, or may be designed, captured, simulated, or documented electronically using various tools, such as Computer Aided Design (CAD), a hardware description language (e.g., VHDL or AHDL), or a PLD programming language (e.g., PALASM, ABEL, or CUPL).

Programmable logic may be fixed either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), or other memory device. The programmable logic may be fixed in a signal that is transmittable to a computer using any of various communication technologies, including, but in no way limited to, analog technologies, digital technologies, optical technologies, wireless technologies (e.g., Bluetooth), networking technologies, and internetworking technologies. The programmable logic may be distributed as a removable storage medium with accompanying p [symbol] electronic documentation (e.g., shrink wrapped software), preloaded with a computer system (e.g., on system ROM or fixed disk), or distributed from a server or electronic bulletin board over the communication system (e.g., the Internet or World Wide Web). In fact, some embodiments, may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A controller for a blood processing device comprising:
a body configured to dock with a first blood processing device to connect the controller to the first blood processing device, the body also configured to undock with the first blood processing device to disconnect the controller from the first blood processing device;
a processor configured to control the first blood processing device when the controller is docked with the first blood processing device and remotely control the first blood processing device when undocked; and
a user interface configured to display information regarding the first blood processing device and an ongoing apheresis procedure when the controller is docked to the first blood processing device and when the controller is undocked from the first blood processing device

2. A controller according to claim 1, wherein the controller is a portable computer.

3. A controller according to claim 1, wherein the controller and/or the processor is configured to control at least a second blood processing device in addition to the first blood processing device.

4. A controller according to claim 3, wherein the controller is configured to control the at least a second blood processing device when docked to the first blood processing device.

5. A controller according to claim 3, wherein the controller is configured to control the at least a second blood processing device when undocked from the first blood processing device.

6. A controller according to claim 3, wherein the controller is configured to control the at least a second blood processing device remotely.

7. A controller according to claim 3, wherein the user interface is configured to user to select which blood processing device to control.

8. A controller according to claim 3, wherein the body is configured to dock with the second blood processing device to connect the controller to the second blood processing device.

9. A controller according to claim 1, wherein the controller begins to automatically control the blood processing device to which it is docked.

10. A controller according to claim 1, wherein the controller is configured to perform at least one function selected from the group consisting of calibrating the blood processing device, calibrating additional components of a blood processing system, record procedure data, record and track procedure incidents, record donor activity, and record data related to sample collection.

11. A controller according to claim 1, further comprising:
a controller connector located on the body, the controller connector configured to connect with a device connector on the first blood processing device when the controller is docked with the first blood processing device, thereby electrically connecting the controller to the first blood processing device.

12. A controller according to claim 1, wherein the body is configured to fit within a docking portion of the first blood processing device, the docking portion supporting the body when the controller is docked to the first blood processing device, thereby physically connecting the controller to the first blood processing device.

13. A controller according to claim 12, wherein the docking portion includes a recess, the body configured to fit within the recess when docked.

14. A controller according to claim 1, further comprising a data storage device store data relating to an apheresis procedure.

15. A controller according to claim 1, wherein the first blood processing device is an apheresis device.

16. A controller according to claim 1, wherein the connection between the controller and the first blood processing device when docked is a wireless connection.

17. A blood processing device comprising:
a cabinet defining the structure of the blood processing device and housing one or more components of the blood processing device, the cabinet including a docking portion;
a blood component separation device for separating whole blood into one or more blood components;
at least one pump configured to control a flow of whole blood and/or blood components through the blood processing device; and
a controller having a body configured to dock with the docking portion to connect the controller to the blood processing device, the body also configured to undock with the blood processing device to disconnect the controller from the blood processing device, the controller including:
a processor configured to control the blood processing device when the controller is docked with the blood processing device and remotely control the blood processing device when the undocked; and
a user interface configured to display information regarding the blood processing device and an ongoing apheresis procedure when the controller is docked to the blood processing device and when the controller is undocked from the blood processing device.

18. A blood processing device according to claim 17, wherein the controller is a portable computer.

19. A blood processing device according to claim 17, wherein the controller and/or the processor is configured to control at least one additional blood processing device in addition to the blood processing device.

20. A blood processing device according to claim 19, wherein the controller is configured to control the at least one additional blood processing device when docked to the blood processing device.

21. A blood processing device according to claim 19, wherein the controller is configured to control the at least one additional blood processing device when undocked from the blood processing device.

22. A blood processing device according to claim 19, wherein the controller is configured to control the at least one additional blood processing device remotely.

23. A blood processing device according to claim 19, wherein the user interface is configured to allow the user to select which blood processing device to control.

24. A blood processing device according to claim 19, wherein the body is configured to dock with the at least one additional blood processing device to connect the controller to the at least one additional blood processing device.

25. A blood processing device according to claim 17, wherein the controller begins to automatically control a blood processing device to which it is docked.

26. A blood processing device according to claim 17, wherein the controller is configured to perform at least one function selected from the group consisting of calibrating the blood processing device, calibrating additional components of a blood processing system, record procedure data, record and track procedure incidents, record donor activity, am related to sample collection.

27. A blood processing device according to claim 17, further comprising:
a blood processing device connector located within the cabinet; and
a controller connector located within the body of the controller, the controller connector configured to connect with the blood processing device connector when the controller is docked with the blood processing device, thereby electrically connecting the controller to the blood processing device.

28. A blood processing device according to claim 27, wherein the blood processing device connector is located within the docking portion.

29. A blood processing device according to claim 17, wherein the docking portion includes a recess, the body configured to fit within the recess when docked, thereby physically connecting the controller to the blood processing device.

30. A blood processing device according to claim 17, wherein the controller further includes a data storage device configured to store data relating to an apheresis procedure.

31. A blood processing device according to claim 17, wherein the blood processing device is an apheresis device.

32. A blood processing device according to claim 17, wherein the connection between the controller and the first blood processing device when docked is a wireless connection.

33. A method for controlling a blood processing device comprising:
providing a blood processing device comprising:
a cabinet defining the structure of the blood processing device and housing one or more components of the blood processing device, the cabinet including a docking portion; and
a blood component separation device for separating whole blood more blood components;
docking a controller with the blood processing device, the controller having a body configured to dock with the docking portion to connect the controller to the blood processing device, the controller also having a processor and a user interface;
controlling, using the controller and/or the processor, the blood processing device;
undocking the controller from the blood processing device, undocking the controller disconnecting the controller from the blood processing device; and
remotely controlling the blood processing device using the undocked controller.

34. A method according to claim 33, further comprising:
selecting, using the user interface, a second blood processing device; and
remotely controlling the second blood processing device using the controller.

35. A method according to claim 33, further comprising:
docking the controller with a second blood processing device; and
controlling the second blood processing device with the controller.

36. A method according to claim 33, further comprising:
docking a second controller to the blood processing device; and
controlling the blood processing device with the second controller.

37. A method according to claim 33, further comprising:
selecting, using a second user interface on a second controller, the blood processing device; and
remotely controlling the blood processing device using the second controller.

38. A method according to claim 33, wherein the connection between the controller and the blood processing device when docked is at least one selected from the group consisting of a physical connection, an electrical connection and/or a wireless connection.
